(19) United States
(12) Patent Application Publication
(10) Pub. No.: US 2014/0241819 A1
(43) Pub. Date: Aug. 28, 2014

(54) Safety Head Bur

(71) Applicant: C. John Munce, Santa Barbara, CA (US)

(72) Inventor: C. John Munce, Santa Barbara, CA (US)

(21) Appl. No.: 14/192,754

(22) Filed: Feb. 27, 2014

Related U.S. Application Data
(60) Provisional application No. 61/770,146, filed on Feb. 27, 2013.

(51) Int. Cl. A61C 3/02; A61B 17/16

(52) U.S. Cl. CPC A61C 3/02; A61B 17/1615
USPC 407/30

(57) ABSTRACT

A safety head bur with a working end with cutting and/or grinding head, a shaft, and a transition zone, which safety head bur provides enhanced safety when used. The cutting and/or grinding head is adapted to cut and/or grind, and has a rearmost diameter. A shaft is attached to the working end. A transition zone extends rearwardly from the rearmost diameter of the working end to a smaller diameter where the transition zone meets the shaft.

### CROSS REFERENCE TO RELATED APPLICATION

This application claims priority from U.S. Provisional Patent Application No. 61/770,146, filed on Feb. 27, 2013, entitled “SAFETY HEAD BUR”.

### BACKGROUND OF THE INVENTION

The present invention relates generally to burs, and more particularly to a bur with a safety head that helps to prevent the bur from catching on out-strokes or pull strokes during use of the bur for grinding or cutting procedures. Dental burs are cutting and grinding implements used to remove tissue from canals in teeth during certain dental procedures, such as root canal procedures. When used in endodontic procedures, their use includes exploration, deep troughing, enlargement of orifices, and canal navigation. Burs are used in other applications including for cutting, grinding and shaping other types of human and animal tissues including bone. Burs can likewise be used in cutting other organic and non-organic materials including wood, plastic, metal, composites, cement, rock, to mention a few.

When used in cutting and grinding relatively small and delicate structures, such as during root canal treatment, endodontists use burs to shape the pulp cavity in the crown and widen and clean out the channels in the roots so that 100% of the dental pulp and other infected tissue can be removed and later on, the filling material, e.g., gutta percha, can be thoroughly packed into the tooth, and any dental post(s) can be located in the channel to later support the filling and crown that are used to reconstruct the tooth.

As shown in FIG. 1 (prior art), current designs of a dental bur 10 have a cutting and/or grinding head 12 attached to a neck region 14 of the shaft 15. A drill end 16 of the shaft 15 opposite the neck 14 is used to connect to a dental drill or other driving tool (not shown.) The neck 14 and drill end 16 of the shaft 15 are formed in a single piece and is typically formed of stainless steel. FIG. 1 shows an exemplary prior art bur 10 in the process of widening and shaping a root channel RC in a root R of a tooth (with rest of tooth not shown). As is often the case, the root channel RC has narrowing and widening portions, and can include a ledge L. Also, as is often the case, a dental drill may itself create a ledge as shown in FIG. 1. The cutting and/or grinding head 12 is the working end of the bur 10 and can be formed of materials such as stainless steel, titanium nitrate carbide, tungsten carbide, diamond, ceramics, plastic or polymers, or other materials. The working surface 18 of the head can be configured with abrasives or, can be fluted. The shaft 15 is adapted to attach to a dental drill (not shown) for spinning the bur. Depending on the material of the cutting and/or grinding head 12, the cutting and/or grinding head 12 is welded, braised, cemented or otherwise attached to the neck 14. In cases where the cutting and/or grinding head 12 is formed of stainless steel, no welds, braises, cements or other attachments are required. However, regardless of the design, current dental burs 10 invariably have a step 20 formed behind the cutting and/or grinding head 12 where the cutting and/or grinding head 12 attaches to the neck 14. The reason for the step 20 is that when manufacturing burs with cutting and/or grinding heads formed of relatively hard material, such as tungsten carbide, it is difficult to manufacture burs without leaving a step as there is risk of the machining tools cutting into the shaft material where the cutting and/or grinding head attaches to the shaft. As can be seen, the head has a rearmost diameter 22 where the abrasive or cutting surface of the cutting and/or grinding head 12 ends. This rearmost diameter 22 is larger than the neck diameter 24 of the neck 14 where the neck 14 attaches to the cutting and/or grinding head 12. The step 20 is relatively flat and is generally on a plane perpendicular to the longitudinal axis LA of the neck 14 and shaft 16. Even if not perfectly perpendicular to the longitudinal axis LA of the neck 14 and shaft 16, the step 20 constitutes a discontinuity where the cutting and/or grinding head 12 is welded to the shaft, or where a terminal end of the neck enters the head in cases where the cutting and/or grinding head is attached to the neck (not shown.) Indeed, there are numerous ways in which a second material—carbide, for example—can be attached to the stainless steel or dissimilar material portion of a shaft. The carbide-to-stainless weld can be formed above the neck so that the stainless part of the bur is formed so as to have a broad portion with a flat surface onto which the carbide “nugget” is welded, with the flutes being ground into both the carbide and the stainless steel portions of what is called the “bur ball”. It is also possible to make the burs with the weld at a point about 3-4 mm down onto the neck 14 or anywhere else on the neck. It is also possible for the burs to be formed entirely from carbide.

During use of prior art burs 10, the step 20 of the bur 10 can often get caught on ledges L in the root channel RC or other areas within the tooth on the out-stroke or pull-stroke. In fact, the intended operation of a bur is always to remove material, and in the case of tooth structure, bone or dental restorative materials, removing hard material may create such a ledge L. If the discontinuity on the backside of a prior art bur should catch on such a ledge, this may drive the cutting and/or grinding head in a manner which can lead to the perforation of root R at its root tip RT or its side S or to further undesirable accentuation of a ledge L. Thus, it would be beneficial if there was a dental bur available that removed the potential for burs to get caught on ledges L in root channels RC of the roots R of teeth.

### SUMMARY OF THE INVENTION

The invention is a safety head bur, comprising: a working end which has a cutting and/or grinding head at a front thereof; a shaft having a proximal end and a distal end; and a smooth transition zone having a wider diameter at a front that extends from the working end and narrows down to a smaller diameter at a rear where the transition zone joins to the distal end of the shaft.

The invention further is a safety head bur, comprising: a working end having cutting flutes formed thereon; a shaft having a proximal end and a distal end; and a frustum-shaped smooth transition zone having a wider diameter at a front that extends from the working end and narrows down to a smaller diameter at a rear where the transition zone joins to the distal end of the shaft.

The invention is yet further a safety head bur, comprising: a working end having a cutting head with flute formed thereon, the cutting head having a rearmost diameter, the cutting head being formed from at least one of the materials selected from the group consisting of stainless steel, titanium nitrate carbide, tungsten carbide, diamond, ceramics, plastic, and polymers; a shaft to which the cutting a head is attached, the shaft being made from material selected from the group consisting of stainless steel, titanium, ceramics, plastic, and polymer; and a frustum-shaped transition zone that extends rearwardly from the rearmost diameter of the cutting head to a smaller diameter where the transition zone meets the shaft.

### BRIEF DESCRIPTION OF THE DRAWINGS

FIG. 1 is a partially exposed side view of a typical prior art carbide dental bur in situ in the root channel of a root of tooth.

FIG. 2 is a partially exposed side view of a first exemplary embodiment of a safety head bur of the invention with a frustum-shaped relief on the back side of the head where it joins with the neck area of the shaft, with the safety head bur in situ in the root channel of a root of a tooth.

FIG. 3 is a side view of a further exemplary embodiment of a safety head bur of the invention.

FIG. 4 is a detail view of a further embodiment of a safety head bur of the invention.

FIG. 5 is a detail view of yet another embodiment of a safety head bur of the invention.

FIG. 6 is a side view of another exemplary embodiment of a safety head bur of the invention.

FIG. 7 is a side detail view of yet another exemplary embodiment of a safety head bur of the invention.

FIG. 8 is a side detail view of a further exemplary embodiment of a safety head bur of the invention.

### DETAILED DESCRIPTION

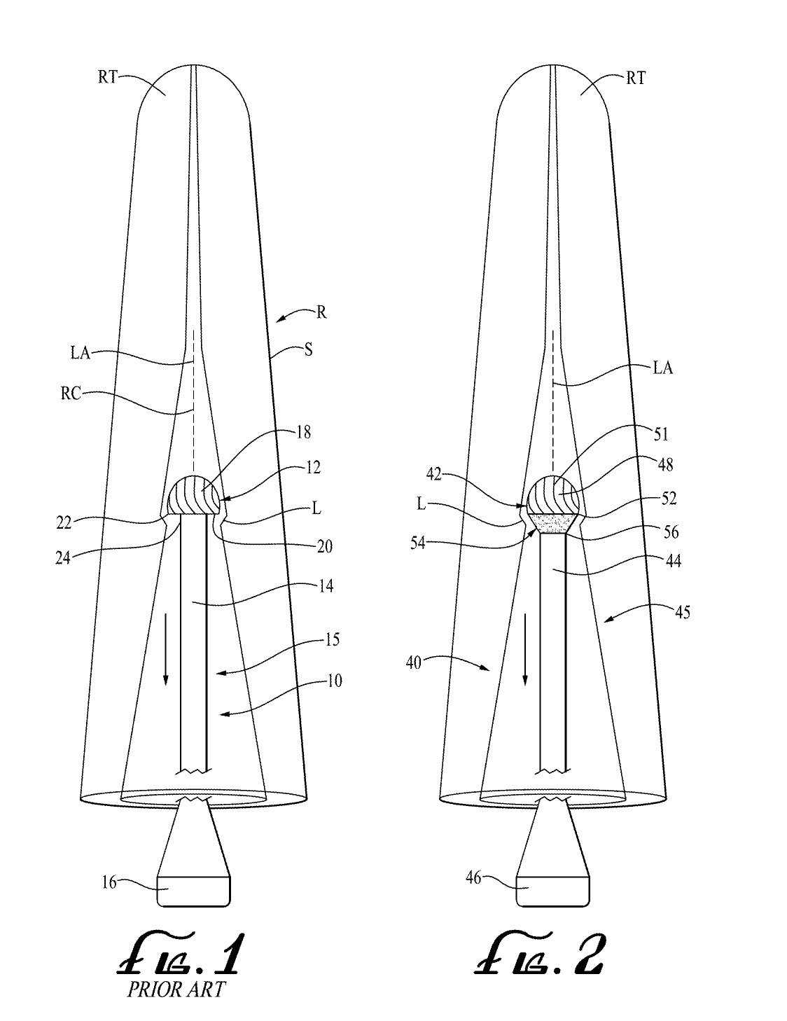

Turning now to the invention, FIG. 2 is a partially exposed side view of a first exemplary embodiment of a safety head bur 40 of the invention in the process of widening and shaping a root channel RC in a root R of a tooth (with rest of tooth not shown). While the safety head bur 40 is well-adapted for use in dental procedures such as cutting, grinding and shaping tooth structure, tooth filling materials, root canal filling materials, alveolar bone, root canal posts formed of metal, composites, glass fibers, ceramics, plastic or other materials, its design is also useful for other applications including for cutting, grinding and shaping bone and other types of tissue and materials in clinical disciplines other than dentistry as well as in research and industries unrelated to clinical disciplines. The safety head bur of the invention can likewise be used in cutting other organic and non-organic materials including wood, plastic, metal, composites, cement, rock, to mention a few. As with prior art burs, it has a cutting and grinding head 42 attached to a neck region 44 of a shaft 45. A drill end 46 of the shaft 45 opposite the neck 44 is used to connect to a dental drill or other driving tool (not shown.) The neck 44 and drill end 46 of the shaft 45 are formed in a single piece which is typically formed of stainless steel. FIG. 2 shows an exemplary safety head bur 40 in the process of widening and shaping a root channel RC in a root R of a tooth (with rest of tooth not shown). The root channel RC has narrowing and widening portions and a ledge L. The cutting and/or grinding head 42 is the working end of the safety head bur 40 and can be formed of materials such as stainless steel, titanium nitrate carbide, tungsten carbide, diamond, ceramics, plastic or polymers, or other materials. A working surface 48 of the cutting and/or grinding head 42 will thus either have an abrasive surface or, in the case of stainless steel, tungsten carbide and certain other materials can be fluted with flutes 51. The shaft 45 is adapted to attach to a dental drill (not shown) for spinning the safety head bur. Depending on the material of the cutting and/or grinding head 42, it may be welded, braised, cemented or otherwise attached to the neck 44. In cases where the cutting and/or grinding head 42 is formed of stainless steel, no welds, braises, cements or other attachment methods are required. Likewise, if a bur is formed entirely of one type of material, carbide for example, no welds, braises, cements or other attachment methods are required. Unlike prior art burs 10 as shown in FIG. 1, there is no step formed behind the cutting and/or grinding head 42 where the cutting and/or grinding head 42 attaches to the neck 44. As can be seen, the head has a rearmost diameter 52 where the abrasive or cutting surface of the cutting and/or grinding head 42 ends, and a smooth frustum-shaped ramp zone 54 transitions from the larger diameter 52 to a smaller neck diameter 56 of the neck 44 where the neck 44 extends from the cutting and/or grinding head 42. The frustum-shaped ramp area 54 is smooth and free from any sharp edges or angles that would tend to get the cutting and/or grinding head 42 caught on any obstructions, such as getting caught on a ledge L in a root channel RC of a root R of a tooth, as best shown in FIG. 2 during the out-stroke or pull-stroke of using the safety head bur 40. The carbide-to-stainless weld (or whatever materials are being attached to each other by whatever means) need not be exactly at the equator of the bur ball. The weld can be located exactly at the height of contour, or it can be above or below the height of contour.

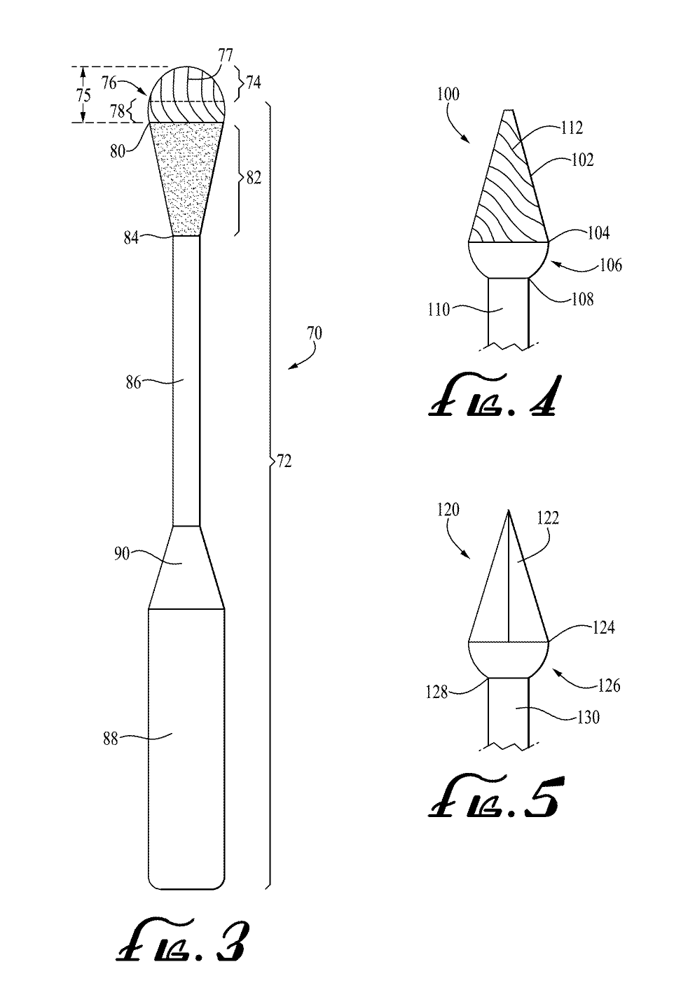

Turning to FIG. 3, there is shown another exemplary embodiment of the safety head bur 70 where an extended shaft 72 is made of a first material, such as stainless steel, and a working end 75 that includes a cutting and/or grinding head 74 made from another material, such as tungsten carbide, and a cutting and/or grinding rear portion 78 behind the cutting and/or grinding head 74 attached by a weld or braise line 76. The cutting and/or grinding rear portion 78 can be made of the same material as the extended shaft, e.g., stainless steel, and comprise a distal end of the extended shaft 72. The cutting and grinding rear portion preferably has flutes 77 or grinding material and such flutes or grinding material may extend partially or completely to a rearmost head diameter 80. Thus, the working end 75 comprises the cutting and/or grinding head 74 and the cutting and/or grinding rear portion 78. A frustum-shaped relief 82 extends from the rearmost head diameter 80 and narrows down to a neck diameter 84, which continues to a neck 86, which continues to a drive end 88. As shown in FIG. 3, the frustum-shaped relief 82 can be made considerably long so that the transition from the wider diameter 80 to the narrower neck diameter 84 occurs gradually. An optional step up area 90 is provided to transition from the smaller diameter of the neck 86 to the larger diameter of the drive end 88 as may be needed for the desired application. The drive end 88 is for connection of the bur 70 with dental drills.

In FIGS. 2 and 3, the cutting and/or grinding heads 48 and 74 are shown as being hemispherical. However, they can have other shapes including parabolic, catenoidal and cylindrical (not shown).

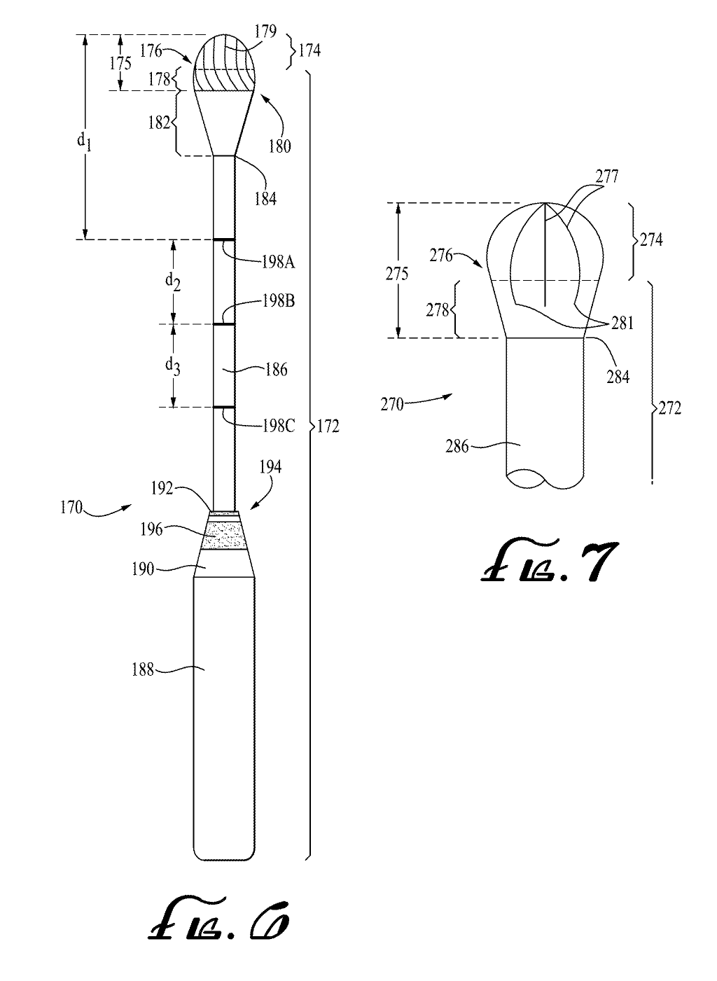

FIG. 4 is a detail view a further embodiment of an exemplary safety head bur 100 of the invention. In this exemplary embodiment, the cutting and/or grinding head 102 is generally cone-shaped and has flutes 112 formed thereon. The rearmost end of the cone-shaped head 102 has a widest rear diameter 104. A convexly-shaped ramp zone 106 extends from the widest rear diameter 104 of the cone-shaped head 102 to narrower diameter 108 where the convexly-shaped ramp zone 106 meets the shaft 110. The convexly-shaped ramp zone 106 will help prevent the bur 100 from getting caught on obstructions on pull-stroke or out-stroke during use. If desired, the ramp zone 106 can also be straight, as in the burs of FIGS. 2 and 3. In FIGS. 4 and 5, the length of the convexly-shaped ramp zone 106 is not shown as being as long as the frustum-shaped relief 82 of the safety head bur 70 of FIG. 3, but it can be of longer if desired.

FIG. 5 is a detail view a further embodiment of an exemplary safety head bur 120 of the invention. In this exemplary embodiment, the cutting and/or grinding head 122 is fluted and generally cone-shaped. The rearmost end of the cone-shaped head 122 has a widest rear diameter 124. A convexly-shaped ramp zone 126 extends from the widest rear diameter 124 of the cone-shaped head 122 to narrower diameter 128 where the convexly-shaped ramp zone 126 meets the shaft 130. The convexly-shaped ramp zone 126 will help prevent the bur 120 from getting caught on obstructions on pull-stroke or out-stroke during use. If desired, the ramp zone 126 can also be straight, as the burs of FIGS. 2 and 3.

FIG. 6 is a side view of another exemplary embodiment of a safety head bur 170 of the invention which is similar to the safety head bur 70 shown and described with reference to FIG. 3. The safety head bur 170 has an extended shaft 172 made of a first material, such as stainless steel, and a working end 175 that includes a cutting and/or grinding head 174 that is made from another material, such as tungsten carbide, and a cutting and/or grinding rear portion 178 behind the cutting and/or grinding head 174 and permanently attached thereto by a weld or braise line 176. The cutting and/or grinding rear portion 178 can be made of the same material as the extended shaft, e.g., stainless steel, and comprise a distal end of the extended shaft 172. The cutting and/or grinding rear portion preferably has flutes 179 or grinding material and such flutes 179 or grinding material may lie behind the weld or braise line 176 and extends partially or completely to a rearmost head diameter 180. Thus, the working end 175 comprises the entire cutting and/or grinding head including working end 174 and the cutting and/or grinding rear portion 178. A frustum-shaped relief 182 extends from the rearmost head diameter 180 and narrows down to a neck diameter 184, which continues to a neck 186, which continues to a drive section 188. The frustum-shaped relief 182 can be made considerably long so that the transition from the wider diameter 180 to the narrower neck diameter 184 occurs gradually. An optional step up area 190 is provided to transition the smaller neck 186 diameter to the larger diameter of the drive section 188 as may be needed for the desired application. The drive section 188 is for connection of the bur 170 with dental drills. As so far, the safety head bur 170 is like the safety head bur 70 of FIG. 3. The differences are now described. The safety head bur 170 includes a shelf or ledge 192 at the transition 194 between where the neck 186 joins to the drive section 188. Optionally, a color coded sleeve 196 can be located either on the neck 186, on the step up area 190 or on the drive section 188 (not shown). The color coding can be made to correlate with information about the safety head bur 170, such as the nature of its 170 cutting head cutting and/or grinding head 175 and/or the frustum-shaped relief 182. The shelf 192 can be very narrow, and will act as a positive stop, such as when sliding cotton pliers are used to insert the drive section 188 into a rotating handpiece (not shown). The shelf 192 will assist with the insertion process and will also help prevent abrasion of the color coded sleeve 196 by the cotton pliers during insertion, if the color coded sleeve is located either on the step up area 190 or on the drive section 188. Lastly, a series of visually identifiable stripes 198A, 198B, and 198C can be optionally located on the neck 186, wherein stripe 198A is located a distance d₁ from the very tip of the working end 175, stripe 198B is located a distance d₂ from the stripe 198A, and stripe 198C is located a distance d₃ from the stripe 198B. Distances d₁, d₂, and d₃ can be the same or different, and will provide a visual guide to aid the practitioner in determining how deep the cutting head and/or grinding head 175 and the frustum-shaped relief 182 are in a tooth being treated. The particular design of the cutting head and/or grinding head and the frustum-shaped relief can be modified as desired, e.g., to have the shapes as described with respect to FIGS. 2, 4, and 5 or other desired shapes and configurations.

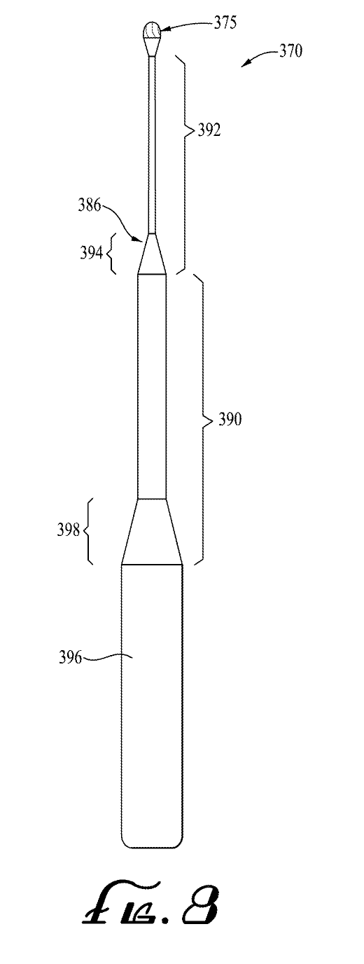

Turning next to FIG. 7, there is shown a side detail view of yet another exemplary embodiment of a safety head bur 270 of the invention which is similar to the safety head burs 70 and 170 shown and described with references to FIGS. 3 and 6, respectively. The balance of the extended shaft 272 behind a neck diameter 284 is not shown, but can have the features as otherwise shown and described in the embodiments FIGS. 3 and 6. The safety head bur 270 has an extended shaft 272 made of a first material, such as stainless steel, and a working end 275 whose cutting and/or grinding head 174 is made from another material, such as tungsten carbide. Unlike the embodiment of the safety head bur 70 and 170 of FIGS. 3 and 6, respectively, in this embodiment, the working end 275 comprises a cutting and/or grinding head 274 at a front thereof, and a generally frustum-shaped cutting and/or grinding rear portion 278 that is welded to the cutting and/or grinding head 274 by a weld or braise line 276. The generally frustum-shaped cutting and/or grinding rear portion 278 is preferably formed of the same material as the extended shaft 272 and can comprise a distal end thereof. The generally frustum-shaped cutting and grinding rear portion 278 has flutes 277 formed thereon, which flutes 277 continue from the cutting and/or grinding head 274 and progress backward of the weld or braise line 276, dying into the frustum-shaped cutting and/or grinding rear portion 278 as the flutes 277 approach the narrowed neck diameter 284 (shown terminating at points 281), which continues to a neck 286, and continues to a drive section (not shown.) The generally frustum-shaped cutting and grinding rear portion 278 can be made longer if desired. Thus, on an outstroke of the safety head bur 270, the flutes 277 on the generally frustum-shaped cutting and grinding rear portion 278 do cut or grind, but do so in a controllable manner so as not to catch in the root channel. The result is that the design of safety head bur 270 resists veering into the body of the tooth and inadvertently causing unintended cutting or grinding of the tooth.

Referring lastly to FIG. 8, there is shown an additional detail of an embodiment of a safety head bur 370 wherein the neck 386 narrows in diameter from a wider proximal neck section 390 to a narrower distal neck section 392 that joins to the working end 375. The transition from the wider proximal neck section 390 to the narrower distal neck section 392 can occur over a transition area 394, or can just be a step down in diameter (not shown.) The larger diameter of the shaft 396 can narrower over a transition area 398 to the narrower diameter of the wider proximal neck section 390. In cases of safety head burs 370 with small working ends 375, incorporating a narrower distal neck section 392 can improve contact of the working end 375 with tissue to be cut or ground away without contact with the neck 386. The feature of having a narrowing neck 386 can be incorporated into the other embodiment shown and described herein.

The preferred embodiments of this invention have been disclosed. However, so that one of ordinary skill in the art would recognize that certain modifications would come within the scope of this invention, it is, therefore, to be understood that within the scope of the appended claims, the invention may be practiced otherwise than as specifically described.

What is claimed is:

1. A safety head bur, comprising:
a working end which has a cutting and/or grinding head at a front thereof;
a shaft having a proximal end and a distal end; and
a smooth transition zone having a wider diameter at a front that extends from the working end and narrows down to a smaller diameter at a rear where the transition zone joins to the distal end of the shaft.

2. The safety head bur of claim 1, wherein the transition zone is frustum-shaped.

3. The safety head bur of claim 1, wherein the transition zone transitions convexly from its front to its rear.

4. The safety head bur of claim 1, wherein the cutting and/or grinding head is formed from at least one of the materials selected from the group consisting of stainless steel, titanium nitrate carbide, tungsten carbide, diamond, ceramics, plastic, and polymers.

5. The safety head bur of claim 1, wherein the cutting and/or grinding head is one of generally spherical or conical in shape.

6. The safety head bur of claim 1, wherein the shaft is made from material selected from the group consisting of stainless steel, titanium, ceramics, plastic, carbide, and polymers.

7. The safety head bur of claim 1, wherein the cutting and/or grinding head of the working end is made of a first material and is attached along a weld or braise line to a cutting and grinding rear portion made of a second material, wherein the second material is the same material as the shaft, and wherein the cutting and grinding rear portion lies behind the weld or braise line and extends to the front of the transition zone.

8. The safety head bur of claim 1, wherein the transition zone is an extension of the shaft.

9. The safety head bur of claim 1, wherein the working end comprises the cutting and/or grinding head at a front thereof made of a first material, and a cutting and/or grinding rear portion made of a second material, which second material is the same material as the shaft, and wherein the cutting and/or grinding head is joined to the cutting and grinding rear portion by welding or braising.

10. The safety head bur of claim 9, wherein the cutting and grinding rear portion comprises the transition zone.

11. The safety head bur of claim 1, wherein flutes are formed on the working end.

12. The safety head bur of claim 11, wherein the flutes die off in the working end.

13. The safety head bur of claim 11, wherein flutes partially extend onto the transition zone and die off in the transition zone.

14. The safety head bur of claim 1, wherein at an end opposite of the cutting and/or grinding head the shaft has a drive section, which drive section has a larger diameter than a diameter of a neck portion of the shaft attached to the cutting and/or grinding head.

15. The safety head bur of claim 14, wherein the diameter of the drive section increases from the smaller diameter of the neck portion of the shaft to the larger diameter of the drive section in a step up area.

16. The safety head bur of claim 15, wherein a shelf is located where the shaft transitions to the drive section, which shelf provides a sharp transition in diameter between the shaft and the drive section.

17. The safety head bur of claim 14, wherein the neck portion has a narrower diameter at a distal neck section attached to the working end and a wider diameter at a proximal neck section.

18. The safety head bur of claim 1, further comprising at least one visually identifiable stripe located on the shaft set a predetermined distance from the forward tip of the working end.

19. The safety head bur of claim 1, further comprising color coding to uniquely identify each safety head bur.

20. A safety head bur, comprising:
a working end having cutting flutes formed thereon;
a shaft have a proximal end and a distal end; and
a frustum-shaped smooth transition zone having a wider diameter at a front that extends from the working end and narrows down to a smaller diameter at a rear where the transition zone joins to the distal end of the shaft.

21. The safety head bur of claim 20, wherein the transition zone transitions convexly from a rearmost diameter of the working end to the smaller diameter where the transition zone meets the shaft.

22. The safety head bur of claim 20, wherein the working end is formed from at least one of the materials selected from the group consisting of stainless steel, titanium nitrate carbide, tungsten carbide, diamond, ceramics, plastic, and polymers.

23. The safety head bur of claim 20, wherein the working end is one of generally spherical or conical in shape.

24. The safety head bur of claim 20, wherein the shaft is made from material selected from the group consisting of stainless steel, titanium, ceramics, plastic, carbide, and polymers.

25. The safety head bur of claim 20, wherein the working end comprises a cutting and/or grinding head made of a first harder material that is attached along a weld or braise line to a cutting and/or grinding rear portion made of a second softer material, the second softer material being the same material of the shaft.

26. The safety head bur of claim 25, wherein the cutting and/or grinding rear portion comprises the transition zone.

27. The safety head bur of claim 20, wherein at the distal end of the shaft there is a drive section, which drive section has a larger diameter than a diameter of the shaft, wherein the diameter of the drive section increases from the smaller diameter of the shaft to the larger diameter of the drive section in a step up area.

28. The safety head bur of claim 27, wherein a shelf is located where the shaft transitions to the drive section, which shelf provides a sharp transition in diameter between the shaft and the drive section.

29. The safety head bur of claim 27, where the shaft further comprises a neck portion at the distal end of the shaft, which neck portion which has a smaller diameter distal end and a wider diameter proximal end.

30. The safety head bur of claim 20, further comprising at least one visually identifiable stripe located on the shaft set a predetermined distance from a tip of the working end.

31. The safety head bur of claim 20, further comprising color coding to uniquely identify each safety head bur.

32. A safety head bur, comprising:
a working end having a cutting head with flutes formed thereon, the cutting head having a rearmost diameter, the cutting head being formed from at least one of the materials selected from the group consisting of stainless steel, titanium nitrate carbide, tungsten carbide, diamond, ceramics, plastic, and polymers;
a shaft to which the cutting a head is attached, the shaft being made from material selected from the group consisting of stainless steel, titanium, ceramics, plastic, carbide, and polymer; and
a frustum-shaped transition zone that extends rearwardly from the rearmost diameter of the cutting head to a smaller diameter where the transition zone meets the shaft.

33. The safety head bur of claim 32, wherein the cutting head of the working end is made of a first harder material and is attached along a weld or braise line to a cutting rear portion made of a second softer material, wherein the second material is the same material as the shaft, and wherein the cutting rear portion lies behind the weld or braise line and extends to the front of the transition zone.

34. The safety head bur of claim 32, wherein the cutting head of the working end is made of a first harder material and is attached along a weld or braise line to a cutting rear portion made of a second softer material, wherein the second material is the same material as the shaft, and wherein the cutting rear portion lies behind the weld or braise line and comprises the transition zone.

35. The safety head bur of claim 32, wherein the flutes die off in the working end.

36. The safety head bur of claim 32, wherein flutes partially extend onto the transition zone and die off in the transition zone.

37. The safety head bur of claim 32, further comprising color banding to identify each safety head bur.

38. The safety head bur of claim 32, wherein the shaft comprises a neck section with a narrower diameter distal neck section attached to the working end and a wider diameter proximal neck section.
